(19) United States
(12) Patent Application Publication
(10) Pub. No.: US 2011/0195944 A1
(43) Pub. Date: Aug. 11, 2011

(54) MODIFIED RELEASE EMULSIONS FOR APPLICATION TO SKIN OR VAGINAL MUCOSA

(75) Inventors: Emanuela Mura, Como (IT); Federico Mailland, Como (IT); Daniela Ceriani, Besano (Varese) (IT)

(73) Assignee: POLICHEM SA, LUXEMBOURG (LU)

(21) Appl. No.: 12/998,331

(22) PCT Filed: Sep. 21, 2009
(86) PCT No.: PCT/EP2009/062190

§ 371 Date: Apr. 7, 2011

(30) Foreign Application Priority Data
Oct. 8, 2008 (EP) 08166058.1

(51) Int. Cl. A61K 31/56; A61K 47/32; A61K 47/38; A61K 31/44; A61K 31/422; A61K 31/437; A61K 31/522; A61K 31/4164; A61P 31/04; A61P 31/10; A61P 33/02

(52) U.S. Cl. 514/182; 514/772.6; 514/781; 514/345; 514/376; 514/293; 514/263.37; 514/398; 514/396

(57) ABSTRACT

Modified release oil in water emulsions that delivers drugs to a target tissue. The emulsions according to the present invention contain the lipophilic active ingredient completely dissolved into the hydrophobic internal phase stabilised by a polymeric surfactant. The presence of this polymer around the hydrophobic droplets avoids the migration of the active ingredient into the external hydrophilic phase and, consequently, its re-crystallization.

This invention relates to oil in water emulsions, suitable to be applied on skin or mucosal surfaces, containing a water insoluble active principle completely dissolved into the internal hydrophobic phase stabilized by a polymeric surfactant from the external hydrophilic phase.

The active principle trapped and stabilized into the internal hydrophobic phase does not migrate into the hydrophilic phase and consequently does not re-crystallize even stored at 40° C. for 6 months.

### BACKGROUND OF THE INVENTION

Water insoluble active principles are difficult to be formulated. In the art, it is known that a drug can be dissolved by using a similar solvent. Consequently, a lipophilic drug is solubilized by using an apolar solvent, e.g. an oil.

To be applied on the skin or mucosal surfaces, oil in water or water in oil compositions, in form of oil in water or water in oil creams and lotions, are an attractive way for drugs formulation.

Semisolid emulsions (i.e. creams and lotions) are two-phases compositions in which one phase (the dispersed or internal phase) is finely dispersed in the other (the continuous or external phase). The dispersed phase can be either hydrophobic based (oil in water creams) or aqueous based (water in oil creams). It is known that a cream is oil in water or water in oil depending on the properties of the system used to stabilize the interface between the phases.

It is known that the use of the appropriate surfactant (i.e. surface active ingredient) improves the physical stability of an emulsion, decreasing the contact angle between the apolar and polar surfaces and, consequently, the active ingredient dissolved into a phase. In most pharmaceutical emulsions stabilizing systems are comprised of either ionic and non ionic surfactants. However, surfactant molecules tend to self-associate, forming micellar or lamellar structures, modifying the stability of the emulsion. Over the time, semisolids tend to modify their physical-chemical properties (i.e. viscosity, appearance and homogeneity) and the active ingredient tends to re-crystallize, due to its migration to the other phase modifying its performances, such as homogeneous distribution into the final product and its delivery.

WO 03/084538 teaches to dissolve a water insoluble active ingredient (Ciclopirox Olamine) into an oil in water emulsion where the emulsifying system, composed by Cocamide DEA (coconut fatty acids diethanolammide), sorbitan monostearate and polysorbate-60, is a standard emulsifying system used to stabilize creams and the like. The formulation disclosed by WO 03/084538 has the disadvantage that the active ingredient tends to migrate into the external hydrophilic phase where it re-crystallizes.

Water emulsions based on standard emulsifying systems are also disclosed in US 2008/0075745 and US 2004/0087564.

### DESCRIPTION OF THE INVENTION

The O/W emulsions object of this invention are stabilized by using a polymeric surfactant, which avoid modification of the internal phase.

The term polymeric surfactant identifies a substance composed of molecules characterized by the multiple repetition of one or more species of atoms or groups of atoms (the repeating constitutional units) linked to each other in amounts sufficient to provide physical and chemical characteristics that do not vary markedly with the addition or removal of one or a few of the repeating constitutional units. Polymeric surfactants form supramolecular self-assemblies where individual block copolymers (unimers) are held together by non-covalent interactions (R. Savic et al. J Drug Target, 2006:14(6):343-355).

Use of polymeric surfactants is already known in the art. Stabilizing systems comprise non-ionic polymers e.g. poloxamer block copolymers) or polyelectrolites (e.g polyacrylic/polymethacrylic acids) or mixture of these. Emulsions made by using these molecules are more stable.

Polymers of acrylic acid, such as Pemulen® TR-1 and 2 can be used at very low concentrations (0.2-0.5% (w/w), jellifying around the droplets of the dispersed hydrophobic phase. Generally, these kind of polymers have to be “activated” by using, e.g. sodium hydroxide, potassium hydroxide, ammonium hydroxide, organic amine bases such as triethanolamine, tromethamine, aminomethyl propanol. These polymer activactors have to be already into the water phase before the emulsification step. The activation converts the coiled form to the uncoiled one of the polymers, which organize around the droplet.

In this invention, the use of specific polymeric surfactants at specific concentrations not only stabilizes the physical characteristics of the final product (i.e. phases do not separate or change their viscosity), but, surprisingly, the migration of the active ingredient into the external hydrophilic phase is avoided. Consequently, it does not re-crystallize, even when stored for 6 months at 40° C.

The O/W emulsions according to the present invention are preferably in form of lotions, creams or gels, and are preferably topically applied to the skin or into the vaginal cavity by a suitable applicator.

These emulsions contain an internal hydrophobic phase in amounts ranging between 1 to 40% by weight, preferably from 5 to 30%, more preferably from 10 to 25%, with respect to the weight of the emulsion; said internal hydrophobic phase preferably contains benzyl alcohol and 2-octyldodecanol, more preferably in a ratio ranging from 1:3 to 1:13 by weight, preferably from 1:5 to 1:11, wherein preferred ratios are 1:5, 1:10 or 1:11; the internal hydrophobic phase may also contain other hydrophobic excipients, which are preferably selected from the group comprising medium-chain mono-, di- and triglycerides (i.e. from 6 to 12 carbon atoms mono-, di- and tri-fatty acid esters of glycerol), polyethylene glycol, isopropyl myristate, mineral oils, silicone oils, vegetable oils, such as coconut, cotton seed, peanut, olive, palm, sunflower seed, sesame, corn, soybean oil, or a mixture combination thereof.

The emulsions contain an external hydrophilic phase in amounts ranging between 60 to 99% by weight, preferably from 70 to 95%, more preferably from 75 to 90%, with respect to the weight of the emulsion; said hydrophilic phase preferably contains lower alkanols, polyhydric alcohols, polyethylene glycols, polypropylene glycols or mixtures thereof and, preferably, not more than 80% by weight of purified water, more preferably not more than 60% by weight (according to a particularly preferred embodiment, the water content being from 45% to 60% by weight); the weight percentage is intended with respect to the weight of the hydrophilic phase.

The present O/W emulsions contain at least one polymeric surfactant in amounts ranging from 0.50 to 2.50% by weight, preferably from 1.00 to. 2.00%, with respect to the weight of the emulsion; said polymeric surfactant is preferably selected from the group of acrylates/C₁₀-C₃₀ alkyl acrilate crosspolymers (i.e. high molecular weight copolymer of acrylic acid and a long chain alkyl methacrylate crosslinked with allyl ethers of pentaerythritol) or from the group of cellulose ethers, such as alkylcellulose, preferably methylcellulose, and hydroxyalkylcellulose hydroxypropylmethylcellulose (preferably Methocel® A and K types). Both types of celluloses have a backbone of cellulose but different ratios of hydroxypropyl to methoxyl substitution.

These concentrations are important to obtain a thick layer around the droplet like a “wall” to trap the lipophylic active ingredient. Addition of neutralising agents is not necessary to stabilise the system.

The emulsions may contain a bio/mucoadhesive ingredient in a proportion ranging between 0.5 to 1.5%, with respect to the weight of the emulsion; said bio/mucoadhesive ingredient being selected from the group of Carbomers, dispersed into the hydrophilic phase.

The emulsions also may contain jellifying agents, selected from the groups of semisynthetic celluloses, comprising methylcellulose, carboxymethyl cellulose, hydroxyethyl cellulose, hydroxypropylcellulose, hydroxypropylmethyl cellulose, propylcellulose, polysaccarides gums, such as tragacanth, pectin, carrageenan and guar, alginic acid and its sodium salt and Poloxamers.

The emulsions contain at least a water insoluble active pharmaceutical principle, dissolved into the internal hydrophobic phase, in amounts ranging from 0.01 to 25% by weight, preferably from 0.5 to 15%, more preferably from 1.0 to 10%, with respect to the weight of the emulsion. The active principle is preferably useful in specific and non specific infections (due to e.g. bacteria, fungi and protozoa) or anti-inflammatory drugs. Compositions may also be useful to deliver hormones. The compositions will be prepared according to conventional techniques, and may include compatible excipients and pharmaceutically acceptable carriers, e.g. ionizing agents, antioxidant agents, chelating agents, moisturizing agents, decongestant agents, preservatives, disinfectant and/or antimicrobial agents, flavoring and colorants.

The compositions may also contain, in combination, other active principles with complementary or, in any case, useful activity. Examples of these compositions prepared according to the present invention include: lotion, cream or jellified emulsion.

The pharmaceutical compositions and the uses of the present invention will be described in details by the following examples. It should, however, be noted that such examples are given by way of illustration and not of limitation.

### Example 1

A jellified oil in water emulsion having the following w/w % composition was prepared:

| | | |
|---|---|---|
| 1) | Ciclopirox USP | 0.77% |
| 2) | Polycarbophil¹ | 1.00% |
| 3) | Glycerol USP | 15.00% |
| 4) | Peanut Oil | 9.00% |
| 5) | Acrylates/C10-C30 Alkyl Acrylates² | 1.00% |
| 6) | PEG-8 Caprylic/Capric Glyceride³ | 2.00% |
| 7) | Propylene Glycol USP | 25.00% |
| 8) | 2-Octyl Dodecanol⁴ | 11.00% |
| 9) | Benzyl Alcohol | 1.00% |
| 10) | Purified Water | 34.23% |

¹Noveon AA1 ®;
²Pemulen TR-1;
³Labrasol;
⁴Eutanol G

Cyclopirox was dissolved by magnetic stirring with Benzyl Alcohol and Octyl dodecanol at room temperature. Then, Peanut Oil, Labrasol and Pemulen TR-1 were added and mixed to obtain a homogeneous suspension (Phase A). Glycerol, Propylene Glycol and water were mixed at room temperature. (Phase B). Phase A was added to Phase B at room temperature and mixed at elevated rpm until a homogeneous semisolid formulation was obtained. Polycarbophil was added and the formulation was gently mixed until a homogeneous jellified emulsion was obtained. The obtained jellified emulsion was white and homogeneous in appearance. A light microscopy analysis did not show presence of active ingredient's crystals.

### Example 2

An oil in water lotion formulation having the following w/w % composition was prepared:

| | | |
|---|---|---|
| 1) | Ciclopirox USP | 0.77% |
| 2) | Glycerol USP | 15.00% |
| 3) | Peanut Oil | 9.00% |
| 4) | PEG-6 stearate (and) PEG-32 stearates¹ | 5.00% |
| 5) | Acrylates/C10-C30 Alkyl Acrylates² | 2.00% |
| 6) | Propylene Glycol USP | 15.00% |
| 7) | Mineral Oil | 2.00% |
| 8) | 2-Octyl Dodecanol³ | 9.00% |
| 9) | Benzyl Alcohol | 1.00% |
| 10) | Purified Water | 40.23% |

¹Tefose 1500 ®;
²Pemulen ® TR-1;
³Eutanol G

Cyclopirox was dissolved by magnetic stirring with Benzyl Alcohol and Octyl dodecanol at room temperature. Peanut Oil, Mineral oil and Pemulen TR-1 were added and mixed. Tefose 1500 was melted, added to the previous ingredients and mixed until a homogeneous suspension was obtained (Phase A). Glycerol, Propylene Glycol and water were mixed and lightly heated. (Phase B). Phase A was added to Phase B and mixed at elevated rpm decreasing the temperature until a homogeneous semisolid formulation was obtained. The obtained lotion was white, homogeneous in appearance with a viscosity around 1500 mPas. A light microscopy analysis did not show presence of active ingredient's crystals.

### Example 3

An oil in water cream formulation having the following w/w % composition was prepared:

| | | |
|---|---|---|
| 1) | Nifuratel | 10.00% |
| 2) | Glycerol USP | 15.00% |
| 3) | Almond Oil | 3.00% |
| 4) | PEG-6 stearate (and) glycol | 8.00% |
| | stearate (and) PEG-32 stearates¹ | |
| 5) | Hydroxypropylmethyl cellulose | 2.00% |
| 6) | Propylene Glycol USP | 14.00% |
| 7) | Mineral Oil | 2.00% |
| 8) | 2-Octyl Dodecanol³ | 9.00% |
| 9) | Benzyl Alcohol | 1.00% |
| 10) | Purified Water | 37.00% |

¹Tefose ® 63;
²Methocel ® K100;
³Eutanol G

Nifuratel was dissolved by magnetic stirring with Benzyl Alcohol and Octyl dodecanol at room temperature. Peanut Oil, Mineral oil were added and mixed. Tefose 1500 was melted, added to the previous ingredients and mixed until a homogeneous suspension was obtained (Phase A). Glycerol, Propylene Glycol and water were mixed and lightly heated. Methocel K100 was then dissolved (Phase B). Phase A was added to Phase B and mixed at elevated rpm decreasing the temperature until a homogeneous semisolid formulation was obtained.

The obtained oil in water cream was yellow in colour, due to the presence of Nifuratel, homogeneous in appearance with a viscosity around 1500 mPas. A light microscopy analysis did not show presence of active ingredient's crystals.

### Example 4

A thermosetting gel having the following w/w % composition was prepared:

| | | |
|---|---|---|
| 1) | Ciclopirox USP | 0.77% |
| 2) | PEG 400¹ | 10.00% |
| 3) | Poloxamer 407² | 18.00% |
| 4) | Polycarbophil³ | 1.00% |
| 5) | Hydroxypropylmethyl cellulose⁴ | 1.00% |
| 6) | Propylene Glycol USP | 20.00% |
| 7) | Mineral Oil | 2.00% |
| 8) | 2-Octyl Dodecanol⁵ | 5.00% |
| 9) | Benzyl Alcohol | 1.00% |
| 10) | Purified Water | 41.23% |

¹Lutrol E400;
²Lutrol F127;
³Noveon AA1 ®;
⁴Methocel ® K100;
⁵Eutanol G

The formulation was prepared by using the same method described for Example 4. The obtained gel was white and homogeneous in appearance. A light microscopy analysis did not show presence of active ingredient's crystals.

### Example 5

A thermosetting gel having the following w/w % composition was prepared:

| | | |
|---|---|---|
| 1) | Ciclopirox USP | 0.77% |
| 2) | Poloxamer 407¹ | 18.00% |
| 3) | Polycarbophil² | 1.00% |
| 4) | Acrylates/C10-C30 Alkyl Acrylates³ | 1.00% |
| 5) | Propylene Glycol USP | 20.00% |
| 6) | Isopropyl Myristate⁴ | 5.00% |
| 7) | 2-Octyl Dodecanol⁵ | 10.00% |
| 8) | Benzyl Alcohol | 1.00% |
| 9) | Purified Water | 43.23% |

¹Lutrol F127;
²Noveon AA1 ®;
³Pemulen TR-1;
⁴Crodamol IPM;
⁵Eutanol G

The formulation was prepared by using the same method described for Example 1. The obtained gel was white and homogeneous in appearance. A light microscopy analysis did not show presence of active ingredient's crystals.

### Example 6

A jellified oil in water emulsion having the following w/w % composition was prepared:

| | | |
|---|---|---|
| 1) | Estradiol | 0.03% |
| 2) | Polycarbophil¹ | 1.00% |
| 3) | Glycerol USP | 15.00% |
| 4) | Peanut Oil | 9.00% |
| 5) | Acrylates/C10-C30 Alkyl Acrylates² | 1.00% |
| 6) | PEG-8 Caprylic/Capric Glyceride³ | 2.00% |
| 7) | Propylene Glycol USP | 25.00% |
| 8) | 2-Octyl Dodecanol⁴ | 11.00% |
| 9) | Benzyl Alcohol | 1.00% |
| 10) | Purified Water | 34.97% |

¹Noveon AA1 ®;
²Pemulen TR-1;
³Labrasol;
⁴Eutanol G

The formulation was prepared by using the same method described for Example 1. The obtained jellified emulsion was white and homogeneous in appearance. A light microscopy analysis did not show presence of active ingredient's crystals.

### Example 7

A thermosetting gel having having the following w/w % composition was prepared:

| | | |
|---|---|---|
| 1) | Imiquimod | 5.00% |
| 2) | Poloxamer 407¹ | 18.00% |
| 3) | Polycarbophil² | 1.00% |
| 4) | Acrylates/C10-C30 Alkyl Acrylates³ | 1.00% |
| 5) | Propylene Glycol USP | 25.00% |
| 6) | 2-Octyl Dodecanol⁴ | 10.00% |
| 7) | Benzyl Alcohol | 1.00% |
| 8) | Purified Water | 39.00% |

¹Lutrol F127;
²Noveon AA1 ®;
³Pemulen TR-1;
⁴Eutanol G

The formulation was prepared by using the same method described for Example 1. The obtain gel was white and homogeneous in appearance. A light microscopy analysis did not show presence of active ingredient's crystals.

### Example 8

A jellified oil in water emulsion having the following w/w % composition was prepared:

| | | |
|---|---|---|
| 1) | Acyclovir | 5.00% |
| 2) | Polycarbophil¹ | 1.00% |
| 3) | Glycerol USP | 15.00% |
| 4) | Peanut Oil | 9.00% |
| 5) | Acrylates/C10-C30 Alkyl Acrylates² | 1.00% |
| 6) | PEG-8 Caprylic/Capric Glyceride³ | 2.00% |
| 7) | Propylene Glycol USP | 20.00% |
| 8) | 2-Octyl Dodecanol⁴ | 11.00% |
| 9) | Benzyl Alcohol | 1.00% |
| 10) | Purified Water | 34.00% |

¹Noveon AA1 ®;
²Pemulen TR-1;
³Labrasol;
⁴Eutanol G

The formulation was prepared by using the same method described for Example 1. The obtained jellified emulsion was white and homogeneous in appearance. A light microscopy analysis did not show presence of active ingredient's crystals.

### Example 9

A jellified oil in water emulsion having the following w/w % composition was prepared:

| | | |
|---|---|---|
| 1) | Metronidazole | 1.00% |
| 2) | Polycarbophil¹ | 1.00% |
| 3) | Glycerol USP | 15.00% |
| 4) | Peanut Oil | 9.00% |
| 5) | Acrylates/C10-C30 Alkyl Acrylates² | 1.00% |
| 6) | PEG-8 Caprylic/Capric Glyceride³ | 2.00% |
| 7) | Propylene Glycol USP | 25.00% |
| 8) | 2-Octyl Dodecanol⁴ | 11.00% |
| 9) | Benzyl Alcohol | 1.00% |
| 10) | Purified Water | 34.00% |

¹Noveon AA1 ®;
²Pemulen TR-1;
³Labrasol;
⁴Eutanol G

The formulation was prepared by using the same method described for Example 1. The obtained jellified emulsion was white and homogeneous in appearance. A light microscopy analysis did not show presence of active ingredient's crystals.

### Example 10

A jellified oil in water emulsion having the following w/w % composition was prepared:

| | | |
|---|---|---|
| 1) | Clotrimazole | 1.00% |
| 2) | Polycarbophil¹ | 1.00% |
| 3) | Glycerol USP | 15.00% |
| 4) | Peanut Oil | 9.00% |
| 5) | Acrylates/C10-C30 Alkyl Acrylates² | 1.00% |
| 6) | PEG-8 Caprylic/Capric Glyceride³ | 2.00% |
| 7) | Propylene Glycol USP | 25.00% |
| 8) | 2-Octyl Dodecanol⁴ | 11.00% |
| 9) | Benzyl Alcohol | 1.00% |
| 10) | Purified Water | 34.00% |

¹Noveon AA1 ®;
²Pemulen TR-1;
³Labrasol;
⁴Eutanol G

The formulation was prepared by using the same method described for Example 1. The obtained jellified emulsion was white and homogeneous in appearance. A light microscopy analysis did not show presence of active ingredient's crystals.

### Example 11

### Microscopy Analysis

Active ingredient solubilisation and absence of crystals after storage was investigated by optical microscopy analysis. The compositions employed were prepared as per Example 1, 3, 5 and 9 and compared to the following oil in water cream:

| | |
|---|---|
| Ciclopirox olamine | 1.0% |
| Cetyl alcohol | 5.75% |
| Stearyl alcohol | 5.75% |
| Octyldodecanol | 5.75% |
| Paraffin, light liquid | 5.75% |
| Coconut fatty acids | 4.0% |
| diethanolamine | |
| Polysorbate 60 | 3.5% |
| Myristyl alcohol | 3.0% |
| Sorbitan monostearate | 1.5% |
| Benzyl alcohol | 1.0% |
| Lactic acid | 0.558% |
| Water, purified | 62.442% |

A small quantity of product was carefully applied on a glass slide and pictures were taken by using a 20× objective lens. An image analysis software was used to compare different pictures taken as soon as the samples were prepared to demonstrate that the active ingredient was dissolved. Pictures were taken also after 3 and 6 months of storage at 40° C. to evaluate re-crystallisation of active ingredient. It was concluded that Examples 1, 3, 5 and 9 contain the active ingredient completely dissolved and it does not re-crystallise even after 6 months at 40° C. To the contrary, even if the oil in water cream showed a complete dissolution of the active at the beginning, after 6 months at 40° C. it was observed that crystals formed in the internal phase.

### Example 12

### Rheological Evaluation

Microstructure properties of formulations have been evaluated by performing:
Rheological flow tests: evaluation of flow curve (flow viscosity) and tixotropy;
Dynamic mechanical tests: evaluation of small periodic deformations into the formulation, which determine breakdown or re-arrangement of structure; in the latter case, dynamic mechanical “strain sweep” test evaluated, under increased strain, the storage modulus G′ (indicator of elastic behaviour) and the loss modulus G″ (measure of the dynamic viscous behaviour). Dynamic viscosity η′ has been studied too as rate of energy dissipation in a viscoelastic material.

### Rheological Flow Tests

The compositions employed were as follows:

Example 1, 4, 5 and 7 compared to a commercial oil in water cream.

Flow curves have been determined by using a Rheostress 600 rheometer, equipped with a cone/plate system (Ø=35 mm, angle=2°), and Peltier temperature control, as below described:

| | | | |
|---|---|---|---|
| step 1 | Controlled-Shear (CS) mode | 0.000 Pa-1000. Pa | 180.00 s |
| step 2 | Controlled-Rate (CR) mode | 0.000 1/s-250.0 1/s | 120.00 s |
| step 3 | CR mode | 250 1/s | 30.00 s |
| step 4 | CR mode | 250 1/s-0.000 1/s | 120.00 s |

Samples were applied to the lower plate by using a plastic spatula to ensure that the formulation shearing did not occur.

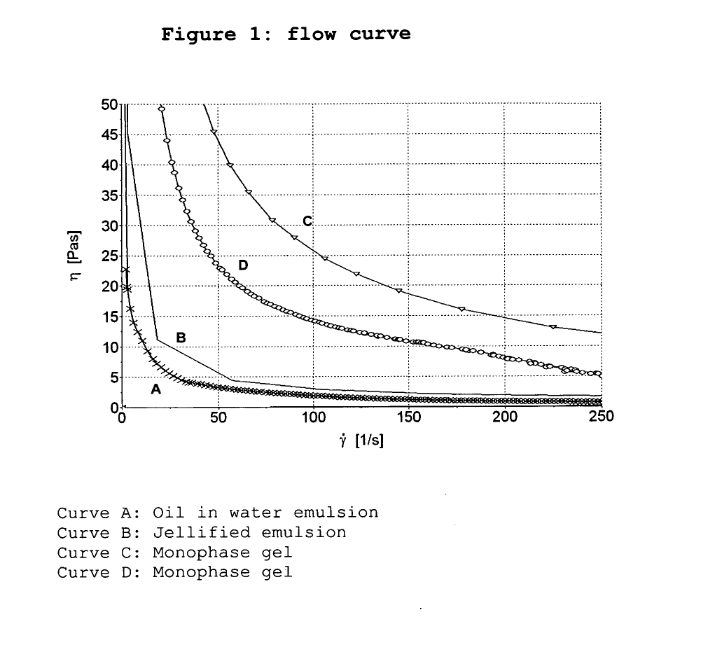

The whole set of measurement have been performed at least in triplicate at a constant temperature of 25° C. The results (see FIG. 1) showed that the jellified emulsion, obtained as per Example 1 (curve B) had a behaviour closer to that of an oil in water emulsion, obtained as per Example 3 (curve A) rather than to those relevant to monophase gels obtained as per Example 5 (curve C) and Example 9 (curve D). Indeed, the high thixotropy value calculated from curve C and D (more than 3×10⁵ Pa s⁻¹) showed a typical scarce structural recovery of the monophase gel structure after stress: the monophase gel network broke. To the contrary, the jellified emulsion thixotropy was low (not more than 1.8×10⁴ Pa s⁻¹) and very close to that of the oil in water emulsion, showing a good structure recovery after stress.

### Dynamic Mechanical Tests

The compositions employed were as follows: Example 1, 4 and 5 compared to a commercial oil in water cream Oscillation stress sweep and Frequency sweep measurements have been performed by using a Rheostress 600 rheometer, equipped with a cone/plate system (Ø=35 mm, angle=2°), and Peltier temperature control as below described:
Amplitude sweep study: sample was exposed to an increasing stress, from 0.000 Pa to 1000 Pa, at a constant frequency of 1 Hz. This test allowed the determination of the linear visco-elastic regime of the sample, and therefore the consequent choice of the stress value to use in the other oscillation tests.
Frequency sweep study: perform in a CS mode, from 100 to 0.1 Hz, at a shear stress selected from the results of amplitude sweep (G′ linear region).

All measurements, have been performed at least in triplicate on each test item and at a constant temperature of 25° C.

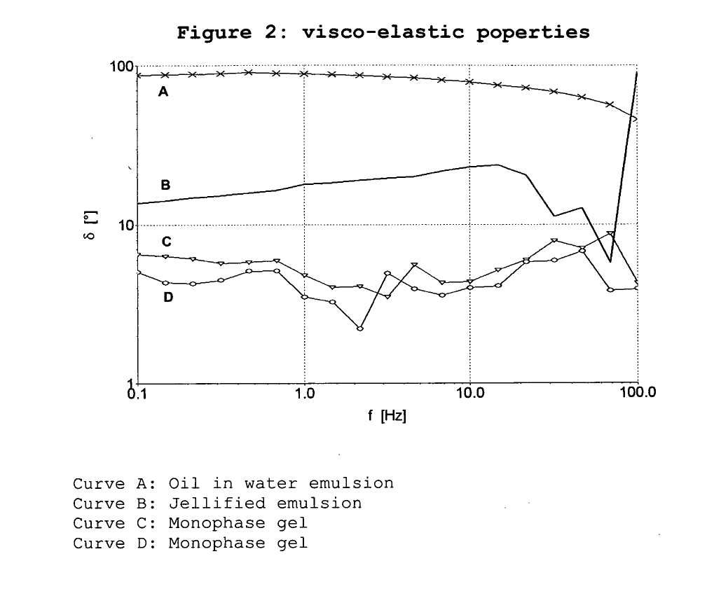

Results are shown in FIG. 2.

The results (see FIG. 2) showed that the jellified emulsion obtained as per Example 1 (curve B) had an intermediate linear visco-elastic behaviour (δ°≈20). Compared to the oil in water emulsion, obtained as per Example 3 (curve A, δ°≈100) and the two monophase gels obtained as per Example 3 and Example 9 (curve C and D respectively, δ°≈5 in both cases), the innermost intermediate structure of the jellified emulsion was confirmed.

### Example 13

### Release Profile

The compositions employed were as follows: Example 1 and 7 compared to a commercial gel.

API's release has been evaluated by using the USP XXIV dissolution apparatus 2 equipped with the Enhancer Cell. Formulation was exactly weighted and packed into the Enhancer Cell. Therefore, only the upper surface of the semisolid was in contact with the dissolution medium phosphate buffer pH 4.5 separated by a GHP disk membrane (pore size: 0.45 μm).

The Enhancer Cell was settled at the bottom of the vessels containing 500 ml of the dissolution medium at a temperature of 37° C. The distance between the cell surface and the stirring paddle (50 rpm) was 2 cm. UV analysis was carried out every 5 minutes at λ=305 nm for a total time of 10 hrs.

The results show that the release of active from formulations object of this inventions is slower than that from a common gel or cream.

### Example 14

### Microscopy Analysis

Active ingredient solubilisation and absence of crystals after storage was investigated by optical microscopy analysis.

The composition of present Example 1 has been compared to the composition of example 1 of WO 03/084538.

A small quantity of the compositions was carefully applied on a glass slide and pictures were taken by using a 20× objective lens and a light polarizer filter.

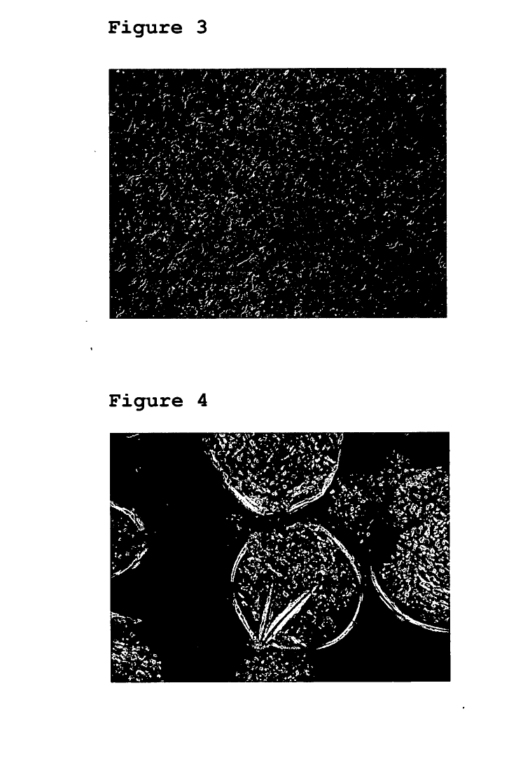

An image analysis software was used to compare different pictures taken as soon as the samples were prepared. FIG. 3 shows the picture of the composition of present Example 1 after 1 week of storage at 50° C.; FIG. 4 shows the picture of the composition of example 1 of WO 03/084538 after 2 hours of storage at 50° C.

Upon comparing the two pictures it can be concluded that the composition according to the present invention contains the active ingredient completely dissolved and it does not re-crystallise after 1 week at 50° C. To the contrary, as regards the composition of example 1 of WO 03/084538, crystals are formed in the internal phase after only 2 hours at 50° C., breaking and destroying the micelle structure of the emulsion.

1-19. (canceled)

20) An oil in water emulsion comprising (i) an internal hydrophobic phase in an amount from 1 to 40% by weight, (ii) an external hydrophilic phase in an amount from 60 to 99% by weight, (iii) at least a polymeric surfactant in an amount from 0.50 to 2.50% by weight and (iv) at least a water insoluble active principle in an amount from 0.01 to 25% by weight, and wherein the polymeric surfactant is selected from acrylates/C₁₀-C₃₀ alkyl acrilate crosspolymers and cellulose ethers.

21) The oil in water emulsion of claim 20, wherein the internal hydrophobic phase is present in an amount from 5 to 30% by weight.

22) The oil in water emulsion of claim 21, wherein the internal hydrophobic phase is present in an amount from 10 to 25% by weight.

23) The oil in water emulsion of claim 20, wherein the internal hydrophobic phase comprises benzyl alcohol and 2-octyldodecanol.

24) The oil in water emulsion of claim 23, wherein the weight ratio of benzyl alcohol:2-octyldodecanol ranges from 1:3 to 1:13.

25) The oil in water emulsion of claim 24, wherein the weight ratio of benzyl alcohol:2-octyldodecanol ranges from 1:5 to 1:11.

26) The oil in water emulsion of claim 20, wherein the external hydrophilic phase is present in an amount from 70 to 95% by weight.

27) The oil in water emulsion of claim 26, wherein the external hydrophilic phase is present in an amount from 75 to 90% by weight.

28) The oil in water emulsion of claim 20, wherein the external hydrophilic phase comprises water in an amount up to 80% by weight.

29) The oil in water emulsion of claim 20, wherein the external hydrophilic phase comprises water in an amount up to 60% by weight.

30) The oil in water emulsion of claim 20, wherein the external hydrophilic phase comprises lower alkanols, polyhydric alcohols, polyethylene glycols, polypropylene glycols or mixtures thereof.

31) The oil in water emulsion of claim 20, wherein the polymeric surfactant is present in an amount from 1.00 to 2.00% by weight.

32) The oil in water emulsion of claim 20, wherein the cellulose ether is selected from alkylcellulose, methylcellulose, hydroxyalkylcellulose, and hydroxypropylmethylcellulose.

33) The oil in water emulsion of claim 20 which does not comprise neutralizing agents.

34) The oil in water emulsion of claim 20, further comprising a bio/mucoadhesive ingredient in an amount from 0.5 to 1.5% by weight.

35) The oil in water emulsion of claim 34, wherein the bio/mucoadhesive ingredient is a Carbomer.

36) The oil in water emulsion of claim 20, wherein the water insoluble active principle is present in an amount from 0.5 to 15% by weight.

37) The oil in water emulsion of claim 36, wherein the water insoluble active principle is present in an amount from 1.0 to 10% by weight.

38) The oil in water emulsion of claim 20, wherein the water insoluble active principle is selected from anti-bacterial drugs, anti-fungal drugs, anti-protozoal drugs, anti-inflammatory drugs and hormones.

39) The oil in water emulsion of claim 20, wherein the water insoluble active principle is selected from Ciclopirox, Nifuratel, Estradiol, Imiquimod, Acyclovir, Metronidazole and Clotrimazole.

40) The oil in water emulsion of claim 20 which is in the form of a lotion, cream or gel.

41) The oil in water emulsion of claim 20 which is in a topical form for application to the skin or into the vaginal cavity.

42) A method for the treatment of vaginal infections and/or vaginal inflammations comprising administering the oil in water emulsion of claim 20 into the vaginal cavity of a human or animal subject exhibiting vaginal infections and/or vaginal inflammations.

43) The method of claim 42, wherein the vaginal infections are of bacterial, fungal or protozoal origin.
